(19) United States
(12) Patent Application Publication
(10) Pub. No.: US 2011/0223617 A1
(43) Pub. Date: Sep. 15, 2011

(54) IMMUNOASSAYS FOR AUTOANTIBODIES IN CARDIOVASCULAR DISEASES

(76) Inventor: Ming Zhang, Brooklyn, NY (US)

(21) Appl. No.: 13/003,332

(22) PCT Filed: Jul. 9, 2009
(86) PCT No.: PCT/US2009/050156

§ 371 Date: May 31, 2011

Related U.S. Application Data
(60) Provisional application No. 61/079,322, filed on Jul. 9, 2008.

(51) Int. Cl. G01N 33/566

(52) U.S. Cl. 435/7.9; 436/501; 435/7.1

(57) ABSTRACT

The present invention relates to the quantitative measurement of auto-reactive antibodies in a patient sample. In particular, the present invention is directed to, inter alia, a method for predicting the degree of cardiovascular injury in a patient following an ischemic event, said method comprising: immobilizing anti-NMHC II antibody on a solid support; adding a lysate of cardiac tissue to the solid support so that antigens in the lysate are captured by the immobilized antibody; adding a biological sample from the patient to said solid support, and incubating said sample for a time sufficient for IgM autoantibodies in the biological sample to bind to antigens in the cardiac tissue lysate; contacting said solid support with an anti-IgM antibody; removing unbound labeled antibodies; and determining the level of anti NMHC II autoantibodies in the biological sample by measuring the amount of labeled anti-IgM antibody bound to the solid support, wherein elevated levels of anti-NMHC II autoantibodies compared to normal individuals at time of patient admission indicates an increased risk of injury. Such methods are useful, inter alia, in the prognosis and monitoring of cardiovascular diseases.

### 1. FIELD OF THE INVENTION

This invention relates generally to autoantibodies, and more particularly, relates to the quantitative measurement of auto-reactive antibodies in a patient sample for determining cardiovascular disease.

### 2. BACKGROUND OF THE INVENTION

In the United States, approximately eight million people present to a hospital emergency room (ER) every year with chest pain suggestive of cardiac origin (Storrow et al. (2000) Ann. Emerg. Med. 35:449), and even more present to their primary care physician. Acute Coronary Syndrome (ACS) presents as a constellation of symptoms such as chest pain, shortness of breath, inability to maintain physical exertion, sense of dread, pain or tingling on the left arm, and may also be accompanied by clinical signs such as altered electrocardiogram and elevation in biochemical markers of necrosis such as cardiac troponin. Chest pain of suspected cardiac origin is often referred to by its clinical description of angina pectoris. Chest pain is the number two reason for emergency room presentation, accounting for about eight percent of all patients.

The chest pain patient presents a diagnostic nightmare for the emergency room physician. On one hand, if the patient really is having a heart attack, early and rapid therapy is crucial to prevent more damage to the heart muscle, and missed diagnosis may result in poor consequences for the patient including death. On the other hand, if the patient is not having a heart attack and the physician keeps the patient in the hospital for a long time performing many diagnostic tests, the patients will consume precious health care resources that could be better spent on others. In fact, it is estimated that diagnosis of chest pain patients represents about $6 billion of wasted resources in the US alone.

Patients presenting with chest pain may be having stable angina, unstable angina, AMI, non-ischemic cardiac problems such as congestive heart failure, or non-cardiac problems such a gastro esophageal reflux disease (GERD). The optimal therapy for each of these patient types and the urgency for therapy is quite different, hence rapid diagnosis and risk stratification has enormous clinical importance.

Until recently, the diagnosis of an MI was done retrospectively. The criteria established by the World Health Organization (WHO) defined MI as any two of the three characteristics of (a) typical symptoms (i.e., chest discomfort), (b) enzyme rise, and (c) typical ECG pattern involving the development of Q-waves (an indication of necrosed myocardium). With these criteria, which were established some years ago, the “enzyme rise” refers to the rise of serum levels of creatine kinase (CK) or its more cardiac specific isoform CK-MB. CK-MB is one of the molecules released from dead cardiac muscle cells and therefore is a serum marker of necrosis. As a heart muscle cell dies as a result of prolonged ischemia, the cell membrane ruptures, releasing the cytosolic contents into the extracellular fluid space, then into the lymphatic system, and from there it enters the bloodstream.

Since the WHO criteria were first promulgated, new biochemical markers of cardiac necrosis have been discovered and commercialized. (For a complete description of many of these markers, see Wu, A. H. B. (ed.) Cardiac Markers, Humana Press ISBN 0-89603-434-8, 1998). The most specific markers of cardiac necrosis so far developed are the cardiac troponins These are proteins which are part of the contractile apparatus of myocardial cells. Two versions, cTnI and cTnT have been commercialized, and shown to be very specific for detection of even small amounts of myocardial damage. The cardiac troponins, similar to CK-MB, are released from dead cardiac muscle cells when the cell membrane ruptures, and are eventually detectable in the blood. Necrosis can certainly occur as a result of a prolonged myocardial ischemia, but can also result from myocardial cell damage from other causes such as infection, trauma, or congestive heart failure. Thus, the observation of an increase in cardiac markers of necrosis alone does not lead to a definitive diagnosis of myocardial infarction.

The cardiac markers described above are excellent markers of necrosis, but are not markers of ischemia. However, there is much confusion in the medical community and in the literature on this point, and it is not uncommon to see references to troponin, CK-MB and myoglobin (another marker of cardiac necrosis) being described as markers of cardiac ischemia. Although it is true that necrosis is always preceded by and is a consequence of ischemia, it is not true that ischemia always leads to necrosis. Therefore these necrosis markers are not necessarily markers of ischemia. For example, stable angina is cardiac ischemia as a result of exercise which will not necessarily lead to necrosis. If the person stops exertion, the demand will fall to the level which can be adequately supplied by the circulation, and the ischemia dissipates, and there is thus no elevation of markers of cardiac necrosis.

Ischemia is the leading cause of illness and disability in the world. Ischemia is a deficiency of oxygen in a part of the body causing metabolic changes, usually temporary, which can be due to a constriction or an obstruction in the blood vessel supplying that part. The two most common forms of ischemia are cardiovascular and cerebrovascular. Cardiovascular ischemia, in which the body's capacity to provide oxygen to the heart is diminished, is the leading cause of illness and death in the United States. Cerebral ischemia is a precursor to cerebrovascular accident (stroke) which is the third leading cause of death in the United States.

The continuum of ischemic disease includes five conditions: (1) elevated blood levels of cholesterol and other blood lipids; (2) subsequent narrowing of the arteries; (3) reduced blood flow to a body organ (as a result of arterial narrowing); (4) cellular damage to an organ caused by a lack of oxygen; (5) death of organ tissue caused by sustained oxygen deprivation. Stages three through five are collectively referred to as “ischemic disease,” while stages one and two are considered its precursors.

Methods adopted for treatment of ischemic heart disease include the dilatation of the obstructed coronary artery by use of an intravascularly inserted balloon, maintenance of blood flow by intravascular insertion of a stent, and dissolution and removal of a thrombus formed in the blood vessel with the use of a thrombolytic agent. With any of such treatments, it is known that as blood flow is restored in the coronary artery, Ca overload or free radicals occur, increasing the region of cellular necrosis.

A current view of the pathogenesis of Ischemia/reperfusion injury (I/R) includes interruption of blood flow, exposure of cells to hypoxic conditions that initiate cellular intracellular changes leading to cell death through the apoptotic and the neurotic pathways. During this period, morphological changes in the cell membrane occur which ultimately lead to complement activation and the perpetuation of the injury beyond that caused by the intracellular processes alone.

There is currently a pressing need for the development and utilization of blood tests able to detect injury to the heart muscle and coronary arteries. Successful treatment of cardiac events depends largely on detecting and reacting to the presence of cardiac ischemia in time to minimize damage.

### 3. SUMMARY OF THE INVENTION

The present invention is directed to a method for the detection of anti-non-muscle myosin heavy chain (NMHC)-II autoantibodies in a biological sample comprising: immobilizing anti-NMHC II antibody on a solid support; adding a biological sample to said solid support, such that the biological sample is in contact with the anti-NMHC II antibody; incubating said sample for a time sufficient for autoantibodies in the biological sample to bind to the immobilized anti-NMHC II antibody; contacting said solid support with a labeled anti-IgM antibody; removing unbound labeled antibodies; and detecting autoantibodies in the biological sample by measuring the amount of anti-IgM antibody bound to the support.

In one embodiment, the biological sample is selected from blood, serum, plasma, saliva, tears, sweat, urine, and peritoneal fluid. In a particular embodiment, the biological sample is plasma.

In one embodiment, the immobilizing step includes coating anti NMHC II A antibody onto wells of a plate. In another embodiment, the incubation period for autoantibodies in the biological sample to bind to the immobilized anti-NMHC II A antibody is at least ten minutes.

In one embodiment, the detectable label is selected from dyes, fluorescers, radiolables, enzymes, chemiluminescers, and photosensitizers. In a particular embodiment, the anti-IgM antibody is labeled with alkaline phosphatase.

In another embodiment, the present invention is directed to a method for the detection of anti-non-muscle myosin heavy chain (NMHC)-II autoantibodies in a biological sample comprising: immobilizing anti-NMHC II A antibody on a solid support; adding a cardiac tissue homogenate or lysate to said solid support, such that the cardiac tissue homogenate or lysate is in contact with the anti-NMHC IIA antibody; adding a biological sample to said solid support, such that the biological sample is in contact with the anti-NMHC IIA antibody; incubating said sample for a time sufficient for autoantibodies in the biological sample to bind to the immobilized anti-NMHC II A antibody; contacting said solid support with a labeled anti-IgM antibody; removing unbound labeled antibodies; and detecting autoantibodies in the biological sample by measuring the amount of anti-IgM antibody bound to the support.

The present invention is directed to a method for predicting the degree of cardiovascular injury in a patient following an ischemic event, said method comprising: immobilizing anti-NMHC II antibody on a solid support; adding a lysate of cardiac tissue to the solid support so that antigens in the lysate are captured by the immobilized antibody; adding a biological sample from the patient to said solid support, and incubating said sample for a time sufficient for IgM autoantibodies in the biological sample to bind to antigens in the cardiac tissue lysate; contacting said solid support with an anti-IgM antibody; removing unbound labeled antibodies; and determining the level of anti NMHC II autoantibodies in the biological sample by measuring the amount of labeled anti-IgM antibody bound to the solid support, wherein elevated levels of anti-NMHC IIA autoantibodies compared to normal individuals at time of patient admission indicates an increased risk of injury.

In one embodiment, the cardiovascular disease is selected from ischaemic heart disease, congestive heart failure, coronary artery disease, carotid artery disease, atherosclerosis, myocardial infarction, hypertension, restenosis, peripheral artery disease, acute coronary syndrome, and stroke. In a particular embodiment, the cardiovascular disease is myocardial infarction.

In one embodiment, the immobilization step includes coating the antibody to the solid support. In another embodiment, the incubation period for IgM autoantibodies in the biological sample to bind to antigens in the cardiac tissue lysate is at least ten minutes.

In one embodiment, the biological sample contains antibody and is selected from the group comprising blood, serum, plasma, saliva, tears, sweat, urine, peritoneal fluid, and other suitable bodily fluids. In a particular embodiment, the biological sample is plasma. In a related embodiment, the plasma is diluted before addition to the solid support. In another embodiment, the antibody-containing biological sample includes cardiac tissue.

In one embodiment of the method of the present invention, a homogenate of cardiac tissue is used instead of or in combination with a lysate of cardiac tissue. In a related embodiment, cardiac tissue is derived from cadavers.

The present invention is directed to a method for predicting clinical outcome following cardiovascular injury in a patient, said method comprising: providing a biological sample from the patient; detecting anti-human non-muscle myosin heavy chain (NMHC)-IIa IgM autoantibody in the biological sample; and comparing the level of anti-human non-muscle myosin heavy chain (NMHC)-IIa IgM autoantibody in the biological sample to the level of said autoantibody in a healthy population without cardiovascular disease, wherein the changed level of said anti-human non-muscle myosin heavy chain (NMHC)-IIa immunoglobulin M autoantibody in the plasma of the patient following cardiovascular disease is indicative of clinical outcome.

In some embodiments, the anti-human immunoglobulin is detectably labeled wherein the label is chosen from dyes, fluorescers, radiolabels, enzymes, chemiluminescers, and photosensitizers. In one embodiment, the enzyme label includes, but is not limited to, alkaline phosphatase.

Various types of immunoassays can be used in performing the methods of the present invention. For example, enzyme linked immunoabsorbent assay (ELISA), fluorescent immunosorbent assay (FIA), immunohistochemistry, chemical linked immunosorbent assay (CLIA), radioimmuno assay (RIA), flow cytometry such as fluorescence activated cell sorting (FACS), Western blot, and immunoblotting. For a review of the different immunoassays which may be used, see: The Immunoassay Handbook, David Wild, ed., Stockton Press, New York, 1994 (incorporated herein by reference).

In one embodiment, the detecting step of the method of the present invention utilizes an anti-human immunoglobulin antibody with a detectable label. In one embodiment, the reactivity of said autoantibody is determined by immunoassay, immunohistochemistry, flow cytometry, or Western blot. In a related embodiment, the immunoassay is an immunometric assay, competitive immunoassay, competitive immunometric assay, or Enzyme Linked Immunosorbent Assay.

In one embodiment, the biological sample contains antibody and comprises cardiac tissue. In another embodiment, the level of anti-non-muscle myosin heavy chain (NMHC)-II autoantibodies is up to two fold different in plasma of a person with cardiovascular disease as compared to the level of said autoantibody in the plasma of control patients without cardiovascular disease. In another embodiment, the level of anti-non-muscle myosin heavy chain (NMHC)-II autoantibodies in plasma of a person with cardiovascular disease is at least about two standard deviation units different from the average level of said autoantibody in the plasma of control patients without cardiovascular disease.

In some embodiments, any of the methods described herein is repeated at least once to monitor the course of the disease and/or to determine the efficacy of a course of treatment.

These, and other objects, features and advantages of this disclosure will become apparent from the following detailed description of the various aspects of the disclosure taken in conjunction with the accompanying examples and claims.

### 4. BRIEF DESCRIPTION OF THE FIGURES

FIG. 1 shows a schematic diagram of the immunoassay for human natural IgM reactive against NMHC-II.

FIG. 2 shows levels of anti-NMHC-II IgM in normal individuals of different ages.

FIG. 3 shows that the level of anti-NMHC-II IgM is independent of gender in normal individuals.

FIG. 4 shows that the level of anti-NMHC-II IgM is independent of race in normal individuals.

FIG. 5 shows temporal changes in levels of auto-reactive IgM against ischemia specific self-antigen and myocardial ischemia-reperfusion injury in patients undergoing cardiac surgery. Specifically, anti-NMHC II IgM levels significantly decreased after release of the aortic cross-clamp compared to preoperative baseline.

FIGS. 6a and 6b show anti-NMHC II natural IgM in normal individuals and patients with cardiovascular disease. Levels of autoimmune natural IgM to NMHC II in normal individuals and MI patients (see FIG. 6A). Plasma samples were collected from fifty normal individuals and twenty nine MI patients. ELISAs were performed as described in Methods.

FIG. 7 shows demographics of normal and myocardial infarction patients.

FIG. 8 shows the correlation between circulation auto-reactive IgM against NMHC-II with Troponin level (primary clinical parameter of myocardial injury). Spearman correlation between these two variables=0.38; p=0.043.

### 5. DETAILED DESCRIPTION OF THE INVENTION

### 5.1. Definitions

It is to be understood that the terminology used herein is for the purpose of describing particular embodiments only and is not intended to limit the scope of the present invention which will be limited only by the appended claims.

Unless defined otherwise, all technical and scientific terms used herein have the same meanings as commonly understood by one of ordinary skill in the art to which this invention belongs. For example, definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Nothing herein is to be construed as an admission that the invention is not entitled to antedate such disclosure by virtue of prior invention.

It must be noted that as used herein and in the appended claims, the singular forms “a”, “an”, and “the” refer to one or to more than one (i.e., to at least one) of the grammatical object of the article, unless the context clearly dictates otherwise. Thus, for example, reference to the “antibody” is a reference to one or more antibodies and equivalents thereof known to those skilled in the art, and so forth.

A prediction of “clinical outcome” as used herein refers to a prediction (e.g., a probability or likelihood) of mortality or death, particularly cardiac death, or the like. The prediction may be directly expressed as a likelihood of occurrence of one or more of these events, or may be indirectly expressed as a numerical value, particularly where those values are to be compiled as data in a clinical trial of a potential thrombolytic therapy. A method for predicting clinical outcome for a patient after said patient has received surgical therapy, said method comprising: providing a biological sample from the patient; detecting anti-human non-muscle myosin heavy chain (NMHC)-II IgM autoantibody in the biological sample; and comparing the level of anti-human non-muscle myosin heavy chain (NMHC)-II IgM autoantibody in the biological sample to the level of said autoantibody in a healthy population without cardiovascular disease, wherein a decrease in the level of anti-human non-muscle myosin heavy chain (NMHC)-II autoantibodies compared to admission levels in said patient indicates a poor clinical outcome.

The term “infarct” or “infarction” means a region of tissue which is dead and non-functional. For example, it is possible to have a brain infarct as a result of a stroke, or a bowel infarct as a result of severe bowel ischemia. A myocardial infarction (MI) is a region of dead heart muscle which is therefore unable to contribute to the pumping function of the heart. The term “heart attack” usually refers to an acute myocardial infarction or AMI, which is the emerging or developing MI, and is the end stage of ACS.

“Ischemia” is the condition of imbalance between oxygen supply and demand. Ischemia can be transitory or continuous. In the case of myocardial ischemia, the oxygen supply is provided by the blood flow in the coronary arteries. The demand may depend on the physical exertion of the person. Thus, ischemia can result from increased demand with a limited supply (e.g.: as a result of increased stress with occluded coronary arteries), or from suddenly restricted supply, as may occur with plaque disruption and thrombus formation in a coronary artery. The first case is often referred to as stable angina. This word “stable” refers to the fact that the angina is reproducible because the restriction in supply is stable (and usually due to stable plaque), and the ischemia can be reversed by simply ceasing the activity. Unstable angina is chest pain which occurs when coronary artery flow is rapidly compromised due to disruption of a plaque (so called unstable plaque) and is inadequate to supply the oxygen demands of the heart during minimal activity. In this case, the ischemia cannot be stopped by ceasing activity, and it may deteriorate to something worse, such as acute myocardial infarction.

Once the blood supply to the myocardium is restricted, the myocardium becomes starved of oxygen, leading to ischemia. In the early stages, the tissue is reversibly ischemic, meaning that with resumption of blood supply the tissue will recover and return to normal function. After a while, the tissue becomes irreversibly ischemic, meaning that although the cells are still alive, if the blood supply is restored, the tissue is beyond salvation, and will inevitably die. Finally, the tissue dies (i.e., becomes necrosed), and forms part of the myocardial infarct. In fact, myocardial infarction is defined as “myocardial cell death due to prolonged ischemia.”

As used herein, the term “ischemic event” means that the patient has experienced a local and/or temporary ischemia due to partial or total obstruction of the blood circulation to an organ.

“Natural IgM” is used herein to refer to an IgM antibody that is naturally produced in a mammal (e.g., a human). The IgM discussed herein comprises autoantibodies to cardiac antigens, in particular, NMHC-II. Unless stated otherwise, anti-NMHC-II antibodies of the IgM isotype are the analyte being measured. Production of natural IgM antibodies in an individual are important in the initial activation of B-cells, macrophages, and the complement system. IgM is the first immunoglobulin synthesized in the humoral immune response.

### 5.2. Methods of the Present Invention

The present invention is based on the observation that natural IgM autoantibodies against NMHC II are present in human blood, and that the levels of these anti-NMHC II autoantibodies were unrelated to age, gender, and race. Further, anti-human non-muscle myosin heavy chain (NMHC)-II autoantibody levels in plasma were significantly decreased compared to preoperative levels in cardiac surgical patients following release of the aortic cross-clamp during surgery. This finding provides a basis for development of screening method to identify patients at increased risk for cardiovascular injury during surgical intervention. Additionally, the invention provides various therapeutic treatments for cardiovascular disease, particularly myocardial infarction and other ischemic events.

The present invention achieves a highly desirable objective, namely providing methods for the diagnostic and prognostic evaluation of subjects with cardiovascular disease. In particular, this is the first demonstration of auto-reactive natural IgM involvement in myocardial ischemia/reperfusion injury in a common clinical scenario in humans. The present invention provides a quantitative immunoassay for their detection, and the use of such quantitative immunoassay for determining if a patient has or is at risk of increased myocardial damage as a result of a cardiovascular disease, and for determining and/or monitoring the efficacy of a selected course of treatment.

Ischemia/reperfusion injury (I/R) injury has been implicated in many pathological conditions such as myocardial infarction (MI), cerebral ischemic events, intestinal ischemia, vascular surgery, transplantation and trauma. The pathogenesis of I/R injury is as follows: for the duration of an interruption of blood flow, cells are exposed to hypoxic conditions that initiate cellular changes, (e.g., free radical generation and kinase activation), which can lead to cell death through the apoptotic and the neurotic pathways. During this period, there are morphological changes in the cell membrane, including the presentation of neo-epitopes that are recognized by natural IgM as pathological. Subsequently, complement is activated, perpetuating the injury beyond that caused by the intracellular processes alone.

The present invention provides screening methods for the diagnostic and prognostic evaluation of cardiovascular disease, for the identification of subjects possessing a predisposition to cardiovascular disease, and for monitoring patients undergoing treatment for cardiovascular disease, based on detecting levels of anti-human non-muscle myosin heavy chain (NMHC)-II immunoglobulin M autoantibody or anti-cardiac myosin immunoglobulin M autoantibody in blood samples of subjects. The invention also provides methods for detecting levels of anti-human non-muscle myosin heavy chain (NMHC)-II immunoglobulin M autoantibody or anti-cardiac myosin immunoglobulin M autoantibody as a diagnostic or prognostic indicator of the degree of cardiovascular injury in a patient following an ischemic event.

The present invention relates to diagnostic evaluation and prognosis of cardiovascular disease by detecting anti-human non-muscle myosin heavy chain (NMHC)-II immunoglobulin M autoantibody or anti-cardiac myosin immunoglobulin M autoantibody of subjects with cardiovascular disease. The detection of a change in the levels of anti-human non-muscle myosin heavy chain (NMHC)-II immunoglobulin M autoantibody or anti-cardiac myosin immunoglobulin M autoantibody compared to normal patients without cardiovascular disease constitutes a novel strategy for screening, diagnosis and prognosis of cardiovascular disease.

The present invention provides for the use of anti-human NMHC II antibody in immunoassays developed by the inventor to detect the presence of anti-human non-muscle myosin heavy chain (NMHC)-II immunoglobulin M autoantibody. Such immunoassays can be utilized for diagnosis and prognosis of cardiovascular disease. For example, such an assay can be used as a method to predict the degree of cardiovascular injury in a patient following an ischemic event. In accordance with the invention, measurement of anti-human non-muscle myosin heavy chain (NMHC)-II immunoglobulin M autoantibody levels in a subject can be used for predicting the clinical outcome following cardiovascular injury in a patient. The monitoring of serum anti-human non-muscle myosin heavy chain (NMHC)-II immunoglobulin M autoantibody levels can be used prognostically to stage progression of the disease.

The invention further relates to assays developed to detect the level of anti-human non-muscle myosin heavy chain (NMHC)-II immunoglobulin M autoantibody in a subject's sample. Such assays include immunoassays. For example, antibodies may be used to quantitatively detect the presence and amount of anti-human non-muscle myosin heavy chain (NMHC)-II immunoglobulin M autoantibody in a subject's sample. The identification of anti-human non-muscle myosin heavy chain (NMHC)-II immunoglobulin M autoantibodies associated with particular cardiovascular diseases provides a basis for immunotherapy of the disease. Any of the isotypes of anti-NMHC II antibodies may be used (i.e. other than anti-NMHC IIa).

The invention further provides for pre-packaged diagnostic kits which can be conveniently used in clinical settings to diagnose patients having cardiovascular disease or a predisposition to developing cardiovascular disease or complications following an ischemic event. The kits can also be utilized to monitor the efficiency of agents used for treatment of cardiovascular disease. In one embodiment of the invention, the kit comprises components for detecting and/or measuring the levels of anti-human non-muscle myosin heavy chain (NMHC)-II immunoglobulin M autoantibody in a sample. In a second embodiment, the kit of the invention comprises components which detect and/or measure the associated antigens in the biological sample.

The present invention is directed to a method for the detection of anti-non-muscle myosin heavy chain (NMHC)-II autoantibodies in a biological sample comprising: immobilizing anti-NMHC II antibody on a solid support; adding a biological sample to said solid support, such that the biological sample is in contact with the anti-NMHC II antibody; incubating said sample for a time sufficient for autoantibodies in the biological sample to bind to the immobilized anti-NMHC II antibody; contacting said solid support with a labeled anti-IgM antibody; removing unbound labeled antibodies; and detecting autoantibodies in the biological sample by measuring the amount of anti-IgM antibody bound to the support.

The present invention is also directed to a method for predicting the degree of cardiovascular injury in a patient following an ischemic event, said method comprising: immobilizing anti-NMHC II antibody on a solid support; adding a lysate of cardiac tissue to the solid support so that antigens in the lysate are captured by the immobilized antibody; adding a biological sample from the patient to said solid support, and incubating said sample for a time sufficient for IgM autoantibodies in the biological sample to bind to antigens in the cardiac tissue lysate; contacting said solid support with an anti-IgM antibody; removing unbound labeled antibodies; and determining the level of anti NMHC II autoantibodies in the biological sample by measuring the amount of labeled anti-IgM antibody bound to the solid support, wherein elevated levels of anti-NMHC II autoantibodies compared to normal individuals at time of patient admission indicates an increased risk of injury.

The present invention is also directed to a method for the detection of anti-non-muscle myosin heavy chain (NMHC)-II autoantibodies in a biological sample comprising: immobilizing anti-NMHC II antibody on a solid support; adding a cardiac tissue homogenate or lysate to said solid support, such that the cardiac tissue homogenate or lysate is in contact with the anti-NMHC II antibody; adding a biological sample to said solid support, such that the biological sample is in contact with the anti-NMHC II antibody; incubating said sample for a time sufficient for autoantibodies in the biological sample to bind to the immobilized anti-NMHC II antibody; contacting said solid support with a labeled anti-IgM antibody; removing unbound labeled antibodies; and detecting autoantibodies in the biological sample by measuring the amount of anti-IgM antibody bound to the support.

The cardiovascular disease is selected from ischemic heart disease, congestive heart failure, coronary artery disease, carotid artery disease, atherosclerosis, myocardial infarction, hypertension, restenosis, peripheral artery disease, acute coronary syndrome, and stroke. In a particular embodiment, the cardiovascular disease is myocardial infarction.

The present invention is directed to a method for predicting clinical outcome following cardiovascular injury in a patient, said method comprising: providing a biological sample from the patient; detecting anti-human non-muscle myosin heavy chain (NMHC)-IIa IgM autoantibody in the biological sample; and comparing the level of anti-human non-muscle myosin heavy chain (NMHC)-IIA IgM autoantibody in the biological sample to the level of said autoantibody in a healthy population without cardiovascular disease, wherein the changed level of said anti-human non-muscle myosin heavy chain (NMHC)-IIa immunoglobulin M autoantibody in the plasma of the patient following cardiovascular disease is indicative of clinical outcome.

The anti-human immunoglobulin is detectably labeled wherein the label is chosen from dyes, fluorescers, radiolabels, enzymes, chemiluminescers, and photosensitizers. In one embodiment, the enzyme label includes, but is not limited to, alkaline phosphatase. Various types of immunoassays can be used in performing the methods of the present invention. For example, enzyme linked immunoabsorbent assay (ELISA), fluorescent immunosorbent assay (FIA), immunohistochemistry, chemical linked immunosorbent assay (CLIA), radioimmuno assay (RIA), flow cytometry such as fluorescence activated cell sorting (FACS), Western blot, and immunoblotting. For a review of the different immunoassays which may be used, see: The Immunoassay Handbook, David Wild, ed., Stockton Press, New York, 1994 (incorporated herein by reference).

The detecting step of the method of the present invention utilizes an anti-human immunoglobulin antibody with a detectable label. In one embodiment, the reactivity of said autoantibody is determined by immunoassay, immunohistochemistry, flow cytometry, or Western blot. In a related embodiment, the immunoassay is an immunometric assay, competitive immunoassay, competitive immunometric assay, or Enzyme Linked Immunosorbent Assay.

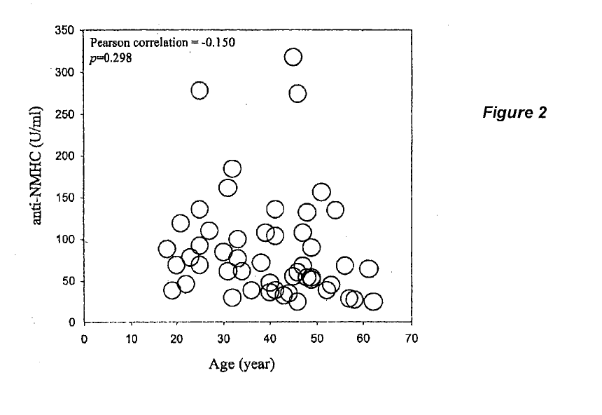

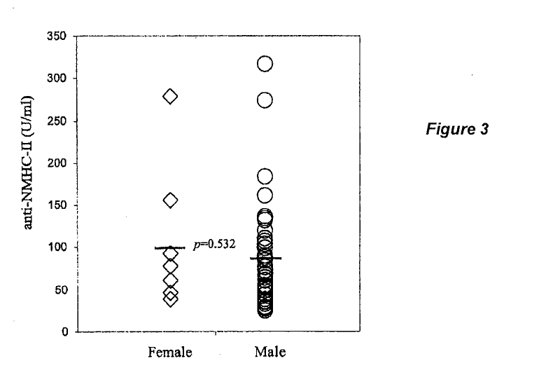

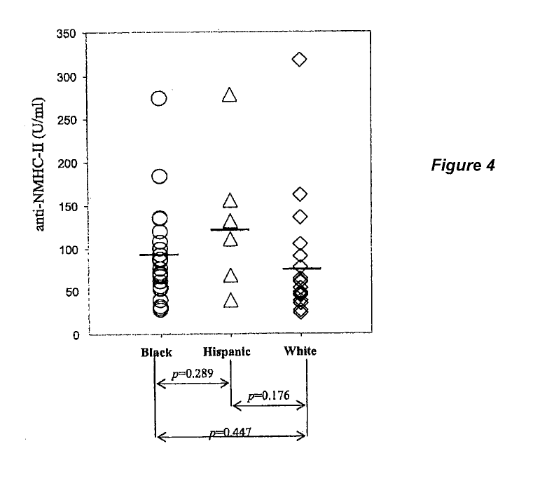

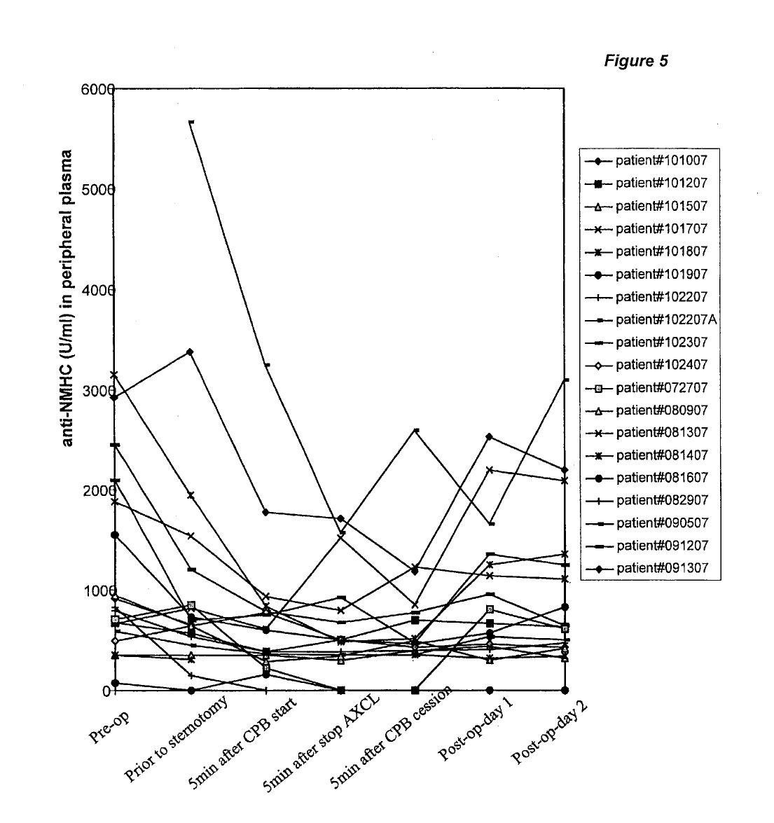

The inventor discovered that IgM against NMHC II is naturally present in the plasma of normal individuals. It has a broad distribution in the tested normal individuals with the highest one being 10-fold more than that of the lowest. Such a distribution was shown to unaffected by age (see FIG. 2), gender (see FIG. 3) or the three races studied (see FIG. 4). The fact that the levels of circulating anti-NMHC-II IgM are independent of these common demographic factors indicates that anti-NMHCI II IgM naturally exist in normal individuals.

Upon presentation by an individual with symptoms of cardiovascular disease, for example myocardial infarction, an assessment of the individual's anti-NMHC-II IgM autoantibody levels at admission to the hospital or clinic enables the clinician to stratify treatment options. A patient with high levels of autoantibodies, for example, may not be appropriate candidate for surgical intervention. Rather, attempts to lower anti-NMHC II levels pre-operatively may be warranted.

Antibodies against various self antigens have been reported in human cardiovascular diseases. For instance, antibodies to cardiac myosin (and actin) were found to increase significantly after MI and correlated with persistent troponin-I elevation at follow-up, late MI, and left ventricular remodeling (Kuch, 1973; Dangas et al., 2000). Other auto-reactive antibodies, i.e. anti-cardiolipin, were also reported to increase after MI, although their correlation with cardiovascular injury is still controversial (De Scheerder et al., 1991; Yilmaz et al., 1994; Bili et al., 2000). The presence of autoantibody against myocardial tropomyosin has been suggested to correlate with the clinical outcome of MI in two reports (Kornilina et al., 1994; Melguizo et al., 1997). However, these studies were impaired by a lack of standardized quantitative measurements of auto-antibodies, and most studies focus on the IgG isotype which is a main player in the adaptive, not the innate immune response. Here, the inventor discovered that anti-NMHC II IgM is naturally present in normal human circulation, developed a quantitative immunoassay to evaluate levels of such a natural IgM, and used this quantitative immunoassay to discover difference in levels of said antibody under different conditions related to a clinical setting.

The first component of the immunometric assay may be added to nitrocellulose or other solid phase support which is capable of immobilizing proteins. By “solid support” is intended any material capable of binding proteins. Well-known solid supports include glass, polystyrene, polypropylene, polyethylene, dextran, nylon, amylases, natural and modified celluloses, polyacrylamides, agaroses, and magnetite. The nature of the support can be either soluble to some extent or insoluble for the purposes of the present invention. The support configuration may be spherical, as in a bead, or cylindrical, as in the inside surface of a test tube or the external surface of a rod. Alternatively, the surface may be flat such as a sheet, test strip, etc. Those skilled in the art will know many other suitable “solid supports” for binding proteins or will be able to ascertain the same by use of routine experimentation. A preferred solid support is a 96-well microtiter plate.

One method in which the antibodies can be detectably labeled is by linking the antibodies to an enzyme and subsequently using the antibodies in an enzyme immunoassay (EIA) or enzyme-linked immunosorbent assay (ELISA), such as a capture ELISA. The enzyme, when subsequently exposed to its substrate, reacts with the substrate and generates a chemical moiety which can be detected, for example, by spectrophotometric, fluorometric or visual means. Enzymes which can be used to detectably label antibodies include, but are not limited to malate dehydrogenase, staphylococcal nuclease, delta-5-steriod isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. One skilled in the art would readily recognize other enzymes which may also be used.

Another method in which antibodies can be detectably labeled is through radioactive isotopes and subsequent use in a radioimmunoassay (RIA). The radioactive isotope can be detected by such means as the use of a gamma counter or a scintillation counter or by autoradiography. Example isotopes include 3 H, 125 I, 131 I, 35 S, and 14 C. One skilled in the art would readily recognize other radioisotopes which may also be used.

It is also possible to label the antibody with a fluorescent compound. When the fluorescent-labeled antibody is exposed to light of the proper wave length, its presence can be detected due to its fluorescence. Among the most commonly used fluorescent labeling compounds are fluorescein isothiocyanate, rhodamine, phycoerythrin, phycocyanin, allophycocyanin, o-phthaldehyde and fluorescamine. One skilled in the art would readily recognize other fluorescent compounds which may also be used.

Antibody can also be detectably labeled by coupling to a chemiluminescent compound. The presence of the chemiluminescent-labeled antibody is determined by detecting the presence of luminescence that arises during the course of a chemical reaction. Examples of chemoluminescent labeling compounds are luminol, isoluminol, theromatic acridinium ester, imidazole, acridinium salt and oxalate ester. One skilled in the art would readily recognize other chemiluminescent compounds which may also be used.

Likewise, a bioluminescent compound may be used to label antibodies. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. One skilled in the art would readily recognize other bioluminescent compounds which may also be used.

Detection of the protein-specific antibody, fragment or derivative may be accomplished by a scintillation counter if, for example, the detectable label is a radioactive gamma emitter. Alternatively, detection may be accomplished by a fluorometer if, for example, the label is a fluorescent material. In the case of an enzyme label, the detection can be accomplished by colorometric methods which employ a substrate for the enzyme. Detection may also be accomplished by visual comparison of the extent of enzymatic reaction of a substrate in comparison with similarly prepared standards. One skilled in the art would readily recognize other appropriate methods of detection which may also be used.

Examples of the solid support include a microplate, a test tube, beads or fine particles made of polystyrene, polyethylene or polyvinyl chloride, a test tube, beads or a filter paper made of glass, or a sheet of dextran, cellulose acetate or cellulose, as well as similar materials thereof. Also, examples of the desirable enzyme to be used in the enzyme immunoassay of the present invention include horseradish peroxidase, alkaline phosphatase, beta-galactosidase and the like. Examples of other assay methods include radioimmunoassay in which a radioactive marker is used, fluoroimmunoassay in which a fluorescent marker is used, chemiluminescence/bioluminescence immunoassay in which a luminescent marker is used and latex agglutination immunoassay in which a latex marker is used.

The binding activity of a given lot of antibodies may be determined according to well known methods. Those skilled in the art will be able to determine operative and optimal assay conditions for each determination by employing routine experimentation.

### 5.3. The Antibody Repertoire and Autoantibodies

Immunoglobulins are high molecular weight proteins which fall into five major classes: IgA, IgD, IgE, IgG and IgM. Collectively, these proteins are commonly referred to as antibodies. Antibodies form, for nearly all higher organisms, the basis of a fundamental immunological defense system against a variety of pathological insults.

A characteristic property of antibodies, regardless of class, is that they function in their defense roles by forming specific complexes with portions of the invading pathogen. This feature of antibodies has been exploited in vitro for a large variety of analytical testing applications such as Radio Immuno Assay (RIA), and Enzyme Linked Immunoassay (ELISA). In-vivo, the specific binding properties of antibodies has been exploited in a large variety of immuno-therapy and imaging techniques.

Over a lifetime, a person confronts the possibility of infection with an almost infinite number of unique foreign substances (antigens). Since it could never be anticipated which of these antigens will ultimately infect a person, it is beneficial that the body possesses an elegant system of producing an equally infinite array of antibodies which recognize, bind and trigger the destruction of antigens.

The monumental repertoire of the adaptive immune system has evolved to allow it to recognize and ensnare virtually any shaped microbial molecule either at present in existence or yet to come. However, in doing so it has been unable to avoid the generation of autoantibodies, antibodies that bind with the body's own constituents and trigger an immunological path of destruction.

Natural immunological tolerance mechanisms prevent the expanded production of autoantibodies. After antibody gene rearrangement, virgin B-cells (the cells that generate antibodies) that display autoantibodies are destroyed or suppressed by the body's tolerance mechanisms. Despite this safety-net, autoantibodies are still produced and for many people create no recognizable pathogenic disorder. It has been estimated that 10-30% of B cells in normal, healthy individuals are engaged in making autoantibodies. Production of autoantibodies is not only the result of an exceptionally diverse immune system, an immune response against one's self can also arise in autoimmune disease or after infections.

### 5.4. Assays for Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly detailed below (but are not intended by way of limitation).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

### 5.5. Diagnostic Tests for Ischemia

A broader array of diagnostic tests are available for diagnosis of ischemia in patients with non-acute symptoms. The EKG exercise stress test is commonly used as an initial screen for cardiac ischemia, but is limited by its accuracy rates of only 25-50%. Coronary angiography, an invasive procedure that detects narrowing in the arteries with 90-95% accuracy, is also utilized. Another commonly used diagnostic test is the thallium exercise stress test, which requires injection of radioactive dye and serial tests conducted four hours apart.

The present invention, however, provides tests for diagnostic and prognostic evaluation of subjects with cardiovascular disease at far lower costs and decreased risk and inconvenience to the patient. Furthermore, the present invention presents a significant time advantage and is cheaper than competing methods of diagnosis.

It is known that immediately following an ischemic event, proteins (enzymes) are released into the blood. Well known proteins released after an ischemic heart event include creatine kinase (CK), serum glutamic oxalacetic transaminase (SGOT) and lactic dehydrogenase (LDH). One well known method of evaluating the occurrence of past ischemic heart events is the detection of these proteins in a patient's blood. U.S. Pat. No. 4,492,753 relates to a similar method of assessing the risk of future ischemic heart events.

However, injured heart tissue releases proteins to the bloodstream after both ischemic and non-ischemic events. For instance, patients undergoing non-cardiac surgery may experience perioperative ischemia. Electro-cardiograms of these patients show ST-segment shifts with an ischemic cause which are highly correlated with the incidence of postoperative adverse cardiac events. However, ST-segment shifts also occur in the absence of ischemia; therefore, electrocardiogram testing does not distinguish ischemic from non-ischemic events. The present invention provides a means for distinguishing perioperative ischemia from ischemia caused by, among other things, myocardial infarctions and progressive coronary artery disease.

### 6. EXAMPLES

The invention, having been generally described, may be more readily understood by reference to the following examples, which are included merely for purposes of illustration of certain aspects and embodiments of the present invention, and are not intended to limit the invention in any way.

6.1. Materials and Methods

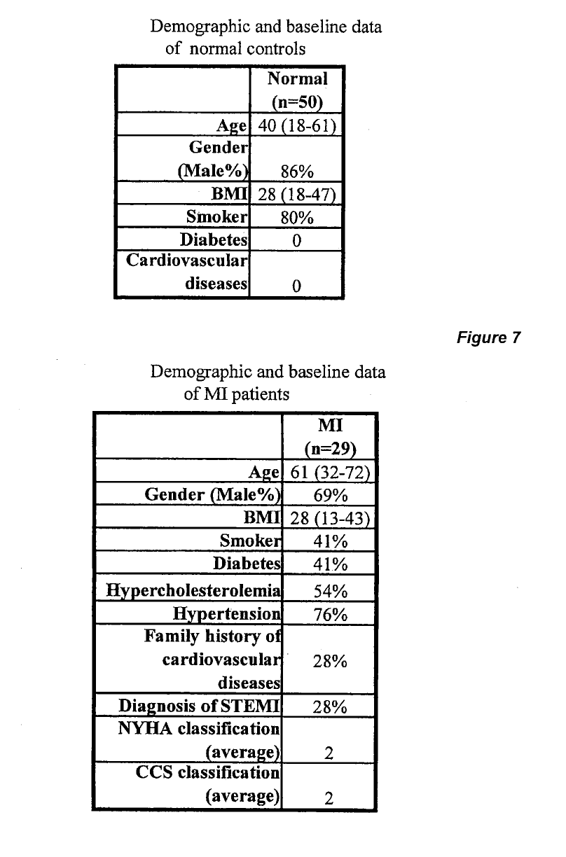

Fifty normal individuals with no history of cardiovascular diseases or diabetes mellitus were selected through the services of Bioreclamation Inc. (Hicksville, N.Y.). Their demographic data is summarized in FIG. 7. Blood was drawn into tubes containing sodium citrate as anti-coagulant (Becton-Dickinson, NJ). After centrifugation, plasma and blood cells were separated and stored frozen at −80° C.

Anti-NMHC-II levels were determined by a sandwich ELISA method. Briefly, Immunolon 2HB microtiter plates (Thermo Scientific, MA) were coated with rabbit IgG antibodies specific for NMHC-II (# MMS-442P, CRP Inc., NJ). The coated wells were washed with Blocking Buffer (PBS, 0.05% Tween-20, 1% BSA-Fraction V, pH7.4) to prevent subsequent non-specific binding. Human myocardial lysate from a cadaver source (National Disease Research Interchange, PA) was added to the coated wells to provide a source of NMHC-II antigen. After thorough washing to remove non-specifically bound molecules, plasma from normal humans (containing natural IgM) was added. The IgM bound to NMHC-II was detected by addition of a commercial anti-IgM antibody conjugated with alkaline phosphatase (SouthernBiotech, AL) and addition of substrate (Sigma, Mo.) producing a colored product detected at 405 nm by a MULTISKAN ASCNT ELISA reader (Thermo Electron Corporation, MA).

To quantify the level of anti-NMHC II IgM in individual human subjects, a range of concentrations of a pooled human plasma sample was used to generate a standard curve. An aliquot of this pooled plasma was included in every experiment to ensure for normalization purposes. The OD obtained when the standard plasma was used undiluted was set at 100 Units/ml (U/ml) and used to convert the ODs obtained from individual plasma samples in this assay to relative concentration values (U/ml).

In the event that the binding efficiency of natural IgM to self antigen is low because the whole plasma has too many proteins, total IgM in patient's plasma is first purified according to standard techniques known in the art, then used in the ELISA as described.

The data were entered into a Microsoft Excel database and analyzed by statistical software (SPSS 16.0, Chicago, Ill.). Associations between age and the level of anti-NMHC II IgM was assessed by 2-tailed Pearson's correlation. Two-sided t-test with was used to analyze the statistical differences between different gender or race groups. Data are expressed as mean±SD and p<0.05 is considered to be statistically significant.

6.2. Results

6.2.1. In Vitro Binding Assay for Anti-NMHC-II Natural IgM

Plasma anti-NMHC-II natural IgM levels were examined by a modified sandwich ELISA method as described supra. In vitro binding assay was performed using the patient's natural IgM which is reactive against non-muscle myosin heavy chain II (NMHC-II). ELISA plates were coated with rabbit IgG antibodies specific for each of the isoforms of NMHC-II (e.g. isoforms A and C on separate plates). Human myocardial lysate from a cadaver source (National Disease Research Interchange, PA) was added to the coated wells to provide NMHC-II antigen. After thorough washing to remove nonspecifically bound proteins, patient's plasma (containing natural IgM) was added. The IgM which binds to NMHC-II was detected by adding commercial anti-IgM antibody conjugated with enzyme (i.e. alkaline phosphatase), followed by substrate for color development at 405 nm.

To quantify the level of autoimmune natural IgM and to normalize the variations among experiments, a solution of pooled human plasma was used as the standard for IgM to generate a standard curve. An aliquot of this standard plasma was included in the assay for use in normalization, and the OD obtained and used to convert ODs obtained from patients' plasma in the assay to concentration values (U/ml).

6.2.2. Changes of Auto-reactive IgM Against Ischemia Specific Self-Antigen and Myocardial Ischemia-reperfusion Injury in Patients Undergoing Cardiac Surgery

Myocardial ischemia occurs as a consequence of aortic cross-clamping and arresting the heart during the normal course of cardiac surgery. Ischemic injury is further enhanced by reperfusion injury to myocardial tissue upon release of the aortic cross-clamp. Ischemia-reperfusion (I/R) injury is a manifestation of the intrinsic cellular response to ischemia and of extrinsic acute inflammation.

Blood samples of 19 cardiac surgical patients were collected at fixed time points in the pre, intra, and post-operative period. Anti-NMHC-II IgM level, troponin level, and routine clinical parameters were analyzed. The results showed that anti-NMHC II IgM levels decreased significantly after release of the aortic cross-clamp (5 min after stop AXCL) compared to preoperative (pre-op) baseline (623+/−533 versus 1192+/−924 U/ml, respectively; p=0.02). Significant increase of troponin level was noted postoperatively compared to preoperative baseline (4.67+/−4.83 versus 0.65+/−1.41 ng/ml, respectively; p=0.001). The increase of troponin level correlated more closely with decrease in anti-NMHC II IgM level (p=0.05) than that of aortic cross-clamp time (p=0.01). Post-operative troponin levels correlated with the decrease in anti-NMHC II IgM level following release of the aortic cross-clamp during surgery (Spearman correlation=0.599, p<0.05). This indicates that auto-reactive IgM antibody against NMHC II may be related to cardiac injury in cardiac surgery.

This is the first study demonstrating auto-reactive natural IgM involvement in myocardial ischemia/reperfusion injury in a common clinical scenario in humans. These data suggest that anti-NMHC II IgM autoantibodies are consumed by binding of the autoantibodies to the newly exposed self-antigen during cardiac surgery following onset of myocardial infarction.

6.2.3. Correlation Between Circulation Auto-Reactive IgM Against NMHC-II with the Admission Peak Troponin Level

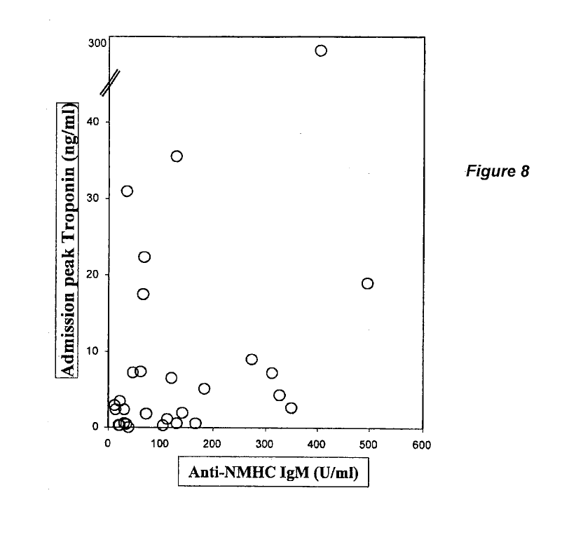

The levels of these autoimmune antibodies were investigated to ascertain whether they correlated with the degree of myocardial injury. The peak admission troponin levels, a known indicator of cardiac injury, was used as the primary parameter for myocardial injury. The average time from admission to the peak level of troponin was 23±17 hrs, and the average value of peak troponin was 16±52 ng/ml (range from 0.04 to 284.4 ng/ml). The Spearman correlation between the anti-NMHC II IgM level and the peak admission troponin level was +0.38 with p=0.043 (see FIG. 8).

This significant correlation between anti-NMHC II IgM and the peak admission troponin level suggested that these autoimmune IgMs attack self targets immediately after myocardial ischemia. These IgMs access the peri-infarct border zone through the collateral circulation, and recognize the exposed self proteins. This is followed by complement activation and tissue damage.

6.2.4. Anti-NMHC II Natural IgM in Normal Individuals and Patients with Cardiovascular Diseases

The inventor discovered that natural IgM antibodies against an ischemia-specific self antigen (non-muscle myosin heavy chain II, NMHC-II) are present in normal individuals. Fifty normal individuals who have no history of diabetes or cardiovascular diseases were recruited through Bioreclamation Inc. (Hicksville, N.Y.). Their demographic data is summarized in FIG. 7.

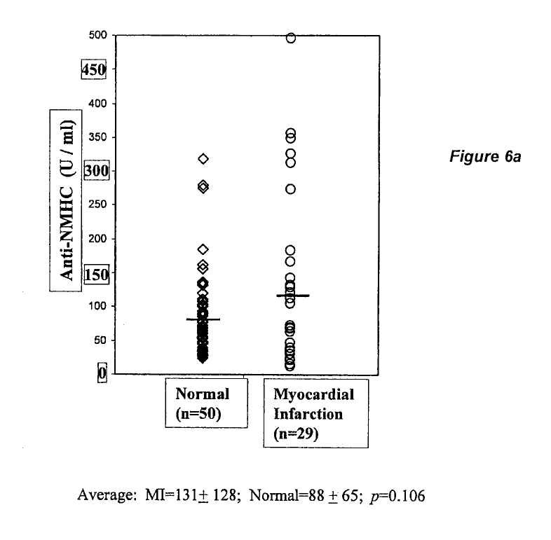

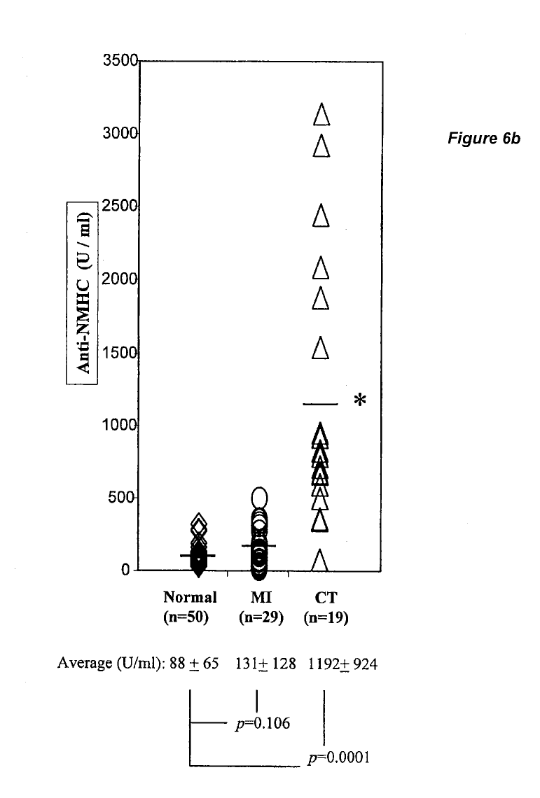

Plasma samples were collected and analyzed by a sandwich ELISA to detect the IgM antibody against NMHC IIA (see FIG. 6). The results showed that the average level of such IgM in normal individuals is 88±65 U/ml, with the highest (318 U/ml) being approximately 10 fold greater than the lowest (24 U/ml). Thus, there is a broad distribution of anti-NMHC II IgM levels among the normal individuals.

FIG. 6 shows the levels of autoimmune natural IgM to NMHC II in normal individuals and MI patients. Plasma samples were collected from fifty normal individuals and twenty nine MI patients. ELISAs were performed as described in Methods. To investigate the anti-NMHC II IgM levels in patients with myocardial infarction, 29 MI patients who agreed to be studied were selected under the approved IRB protocols of Downstate and Lutheran Medical Centers. Patients' demographic and baseline medical data are summarized in FIG. 7. Their plasma levels of anti-NMHC II were analyzed as described for the normal controls (see FIG. 6). IgM levels in these patients ranged, with the average being 131±128 U/ml, the highest being 496 U/ml and the lowest being 12 U/ml.

Comparing the average anti-NMHC II IgM level of MI patients with that of normal individuals showed a 48% increase. This is not statistically significant (p=0.106, by 2-sided t-test with Satterthwaite correction for unequal variances). This is contrast with more than 10 fold increase of anti-NMHC II IgM level in the patients admitted for open heart surgery. One explanation is that patients elected for open heart surgery had much severe cases of heart disease with a long progression compared with the MI patients. Therefore, it is important to evaluate the anti-NMHC II levels in patients elected for cardiac surgery as such antoantibody is related to myocardial injury in cardiac surgery.

6.2.5. In Vitro Competition Assay for the Epitope Recognized by Natural IgM in MI Patient Plasma

Sequences of mouse NMHC-II (all 3 isoforms) share high homology (91100%) with the corresponding sequences of mammals, including humans (see Table 3 infra). We verify that the autoimmune IgM analyzed in the assay recognizes the conserved N2 sequence on NMHC-II in a separate assay. The assay is carried out as described above, but an additional aliquot of the patient's plasma is pre-incubated with the N2 peptide, representing the antigenic epitope on NMHC-II. This blocks the binding of the patient's natural IgM to the human myocardial lysate on the coated well.

TABLE 3
Homology between mouse N2 sequences and human NMHC isoforms

| | |
|---|---|
| Mouse N2 sequences of NMHC-II C | 607-LMKNMDPLNDNV-619 |
| Human NMHC-II A isoforms | 585-LMKNMDPLNDNI-596 |
| (gb\|EAW60098.1\|) | |
| Human NMHC-II B isoforms | 592-LMKNMDPLNDNV-603 |
| (gb\|AAA99177.1\|) | |
| Human NMHC-II C isoforms | 592-LTKNMDPLNDNV-603 |
| (gb\|EAW53924.1\|) | |

References relating to the identification of autoantibodies and their blocking peptides include: Zhang M et al. Natural IgM-mediated innate autoimmunity: a new target for early intervention of ischemia-reperfusion injury. Expert Opin Biol Ther. 2007; 7:1575-82; Zhang and Carroll. Natural antibody mediated innate autoimmune response. Mol Immunol. 2007; 44(1-3):103-10; Zhang et al. The role of natural IgM in myocardial ischemia-reperfusion injury. J Mol Cell Cardiol. 2006; 41:62-7; Zhang et al. Identification of the Target Self-antigens in Reperfusion Injury. J Exp Med. 2006; 203:141-52; Chan et al. Attenuation of skeletal muscle reperfusion injury with intravenous 12 amino acid peptides that bind to pathogenic IgM. Surgery. 2006; 139:236-43; Zhang et al. Identification of a specific self-reactive IgM antibody that initiates intestinal ischemia/reperfusion injury. PNAS. 2004; 101:3886-91; and

Austen et al. Murine hindlimb reperfusion injury can be initiated by a self-reactive monoclonal IgM. Surgery. 2004; 136:401-6, which are incorporated herein by reference in their entireties.

6.2.6. Detection of Total Anti-Heart IgM Autoantibody Levels in Human Blood Sample

Human heart tissue was homogenized, diluted in coating buffer, and added to wells of an ELISA plate. The coated wells were then blocked from non-specific binding by blocking buffer. Individual human plasma samples were then diluted and added to the coated wells. A serial dilution of a reference plasma was made for standard (aliquot from a pooled plasma which was quantitatively compared with a known quantity of monoclonal human IgM, B7). After incubation, auto-reactive IgM bound to the coated heart tissue in the wells was detected by anti-human IgM antibody labeled with alkaline phosphatase (AP). After incubation, substrate for AP was added to color reaction and optical density (OD) was recorded on an ELISA reader. Results were calculated by subtracting the background (blank) and fitting the standard curve.

6.2.7. Detection of Anti-Cardiac Myosin IgM Autoantibody Levels in Human Blood Sample

Anti-human cardiac-myosin antibody (Biogenesis, catalog# 6490-3610) was diluted in coating buffer and added to coat the wells of an ELISA plate. The coated wells were then blocked from non-specific binding by using a blocking buffer. Human heart tissue was then homogenized, diluted in blocking buffer, and added to coated wells. Individual human plasma samples were then diluted and added to the coated wells. A serial dilution of a reference plasma was made for standard (aliquot from a pooled plasma which was quantitatively compared with a known quantity of monoclonal human IgM, B7). After incubation, auto-reactive IgM bound to the captured cardiac myosin from the heart tissue was detected by anti-human IgM antibody labeled with alkaline phosphatase (AP). After incubation, the substrate for AP was added to color reaction and optical density (OD) was recorded on an ELISA reader. Results were calculated by subtracting background (blank) and fitting the standard curve.

6.2.8. Detection of Anti-Non-Muscle Myosin Heavy Chain (NMHC)-II A IgM Autoantibody Levels in Human Blood Sample

Anti-human NMHC II A antibody (CRP Inc., catalog #MMS-442P) was diluted in coating buffer and added to wells of an ELISA plate to coat them. The coated wells were then blocked from non-specific binding by using a blocking buffer. Human heart tissue was homogenized, diluted in blocking buffer, and added to the coated wells. Individual human plasma samples were diluted and added to the coated wells. A serial dilution of a reference plasma was made for standard (aliquot from a pooled plasma). After incubation, auto-reactive IgM bound to the captured non-muscle myosin heavy chain IIA from the heart tissue was detected by anti-human IgM antibody labeled with alkaline phosphatase (AP). After incubation, the substrate for AP was added to color reaction and optical density (OD) was recorded on an ELISA reader. The results were calculated by subtracting background (blank) and fitting the standard curve.

An ELISA-based immunoassay was developed to evaluate anti-NMHC II IgM in normal human plasma. Fifty normal individuals were recruited for this study and their plasma contained anti-NMHC II IgM in a range of 24 to 318 U/ml, with the average being 88±65 U/ml. The difference between the lowest and highest anti-NMHC II IgM was greater than 10-fold.

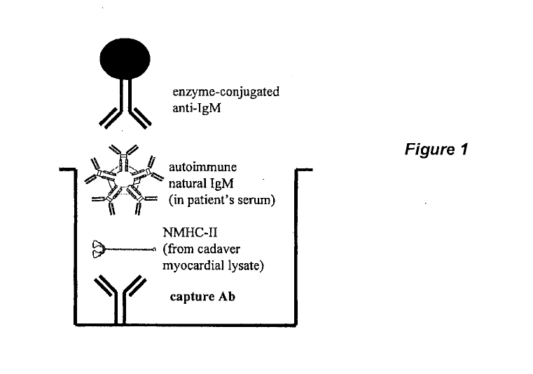

The results showed that anti-NMHC II IgM levels were independent of age (Pearson correlation=−0.150, p>0.05) (see FIG. 2) and did not vary significantly (p>0.05) between males (average=85±62 U/ml) and females (average=107±85 U/ml) (see FIG. 3). Furthermore, there was no statistical difference (p>0.05) between the levels in black (average=89±55 U/ml), white (average=74±68 U/ml) and Hispanic (average=131±84 U/ml) individuals (see FIG. 4). Plasma samples were collected from fifty normal individuals. ELISA was performed as described (see also FIG. 1). Statistical analysis was performed using a 2-sided t test.

All publications, patents, and patent applications mentioned herein are hereby incorporated by reference in their entirety as if each individual publication or patent was specifically and individually indicated to be incorporated by reference. In case of conflict, the present application, including any definitions herein, will control. Those skilled in the art will recognize, or be able to ascertain using no more than routine experimentation, many equivalents to the specific embodiments of the invention described herein. Such equivalents are intended to be encompassed by the following claims.

1. A method for the detection of anti-non-muscle myosin heavy chain (NMHC)-II autoantibodies in a biological sample comprising:
a) immobilizing anti-NMHC II antibody on a solid support;
b) adding a biological sample to said solid support, such that the biological sample is in contact with the anti-NMHC II antibody;
c) incubating said sample for a time sufficient for autoantibodies in the biological sample to bind to the immobilized anti-NMHC II antibody;
d) contacting said solid support with a labeled anti-IgM antibody;
e) removing unbound labeled antibodies; and
f) detecting autoantibodies in the biological sample by measuring the amount of anti-IgM antibody bound to the support.

2. The method of claim 1, wherein said biological sample is selected from blood, serum, plasma, saliva, tears, sweat, urine, and peritoneal fluid.

3. (canceled)

4. The method of claim 1, wherein the immobilizing step includes coating anti NMHC II A antibody onto wells of a plate.

5. The method of claim 1, wherein said incubation period is at least ten minutes.

6. (canceled)

7. (canceled)

8. The method of claim 1, further comprising a step between steps a) and b), wherein a cardiac tissue homogenate or lysate is added to the solid support.

9. A method for predicting the degree of cardiovascular injury in a patient following an ischemic event, said method comprising:
a) immobilizing anti-NMHC II antibody on a solid support;
b) adding a lysate of cardiac tissue to the solid support so that antigens in the lysate are captured by the immobilized antibody;
c) adding a biological sample from the patient to said solid support, and incubating said sample for a time sufficient for IgM autoantibodies in the biological sample to bind to antigens in the cardiac tissue lysate;
d) contacting said solid support with an anti-IgM antibody;
e) removing unbound labeled antibodies; and
f) determining the level of anti NMHC II autoantibodies in the biological sample by measuring the amount of labeled anti-IgM antibody bound to the solid support,
wherein elevated levels of anti-NMHC II autoantibodies compared to normal individuals at time of patient admission indicates an increased risk of injury.

10. The method of claim 9 wherein the cardiovascular injury results from a cardiac disease selected from ischemic heart disease, congestive heart failure, coronary artery disease, carotid artery disease, atherosclerosis, myocardial infarction, hypertension, restenosis, peripheral artery disease, acute coronary syndrome, and stroke.

11. The method of claim 10 wherein the ischemic event is myocardial infarction.

12. The method of claim 9, wherein said biological sample is selected from blood, serum, plasma, saliva, tears, sweat, urine, and peritoneal fluid.

13. (canceled)

14. (canceled)

15. The method of claim 9, wherein the immobilizing step includes coating anti-NMHC II A antibody to wells of a plate.

16. The method of claim 9, wherein said incubation period is at least ten minutes.

17. (canceled)

18. (canceled)

19. The method of claim 9, wherein for step b) a homogenate of cardiac tissue is used instead of or in combination with a lysate of cardiac tissue.

20. The method of claim 9, wherein said cardiac tissue is derived from cadavers.

21. A method for predicting clinical outcome following cardiovascular injury in a patient, said method comprising:
a) providing a biological sample from the patient;
b) detecting anti-human non-muscle myosin heavy chain (NMHC)-II IgM autoantibody in the biological sample; and
c) comparing the level of anti-human non-muscle myosin heavy chain (NMHC)-II IgM autoantibody in the biological sample to the level of said autoantibody in a healthy population without cardiovascular disease,
wherein the changed level of said anti-human non-muscle myosin heavy chain (NMHC)-II immunoglobulin M autoantibody in the plasma of the patient following cardiovascular disease is indicative of clinical outcome.

22. The method of claim 21, wherein said cardiovascular injury results from a cardiac disease selected from ischemic heart disease, congestive heart failure, coronary artery disease, carotid artery disease, atherosclerosis, myocardial infarction, hypertension, restenosis, peripheral artery disease, acute coronary syndrome, and stroke.

23. The method of claim 21, wherein said cardiovascular injury is myocardial infarction.

24. The method of claim 21, wherein said biological sample is selected from blood, serum, plasma, saliva, tears, sweat, urine, and peritoneal fluid.

25. (canceled)

26. The method of claim 21, wherein said detecting step utilizes an anti-immunoglobulin antibody with a detectable label.

27. The method of claim 26, wherein said detectable label is selected from dyes, fluorescers, radiolables, enzymes, chemiluminescers, and photosensitizers.

28. The method of claim 21, wherein the reactivity of said autoantibody is determined by immunoassay, immunohistochemistry, flow cytometry, or Western blot.

29. (canceled)

30. The method of claim 21, wherein said biological sample contains antibody and comprises cardiac tissue.

31. The method of claim 21, wherein the level of said anti-non-muscle myosin heavy chain (NMHC)-II autoantibodies is up to two fold different in plasma of a person with cardiovascular disease as compared to the level of said autoantibody in the plasma of control patients without cardiovascular disease.

32. The method of claim 21, wherein the level of said anti-non-muscle myosin heavy chain (NMHC)-II autoantibodies in plasma of a person with cardiovascular disease is at least about two standard deviation units different from the average level of said autoantibody in the plasma of control patients without cardiovascular disease.
